(19) United States
(12) Patent Application Publication
(10) Pub. No.: US 2025/0352493 A1
(43) Pub. Date: Nov. 20, 2025

(54) HIGHLY ELASTIC PATCHES AND MASKS FOR DELIVERY OF THERAPEUTIC AGENTS

(71) Applicant: TPC-API LLC, FAIRFAX, VA (US)

(72) Inventor: Jordan WITTMAYER, Coronado, CA (US)

(21) Appl. No.: 18/866,779

(22) PCT Filed: May 18, 2023
(86) PCT No.: PCT/US2023/022645

§ 371 Date: Nov. 18, 2024

Related U.S. Application Data
(60) Provisional application No. 63/343,858, filed on May 19, 2022.

(51) Int. Cl. A61K 9/70; A61K 31/00; A61K 31/045; A61K 31/167; A61K 31/4045; A61K 36/185; A61K 36/28; A61K 36/53; A61K 36/9066; A61K 45/06

(52) U.S. Cl. CPC A61K 9/7061; A61K 31/045; A61K 31/167; A61K 31/4045; A61K 31/658; A61K 36/28; A61K 36/3482; A61K 36/53; A61K 36/9066; A61K 45/06

(57) ABSTRACT

The subject invention relates to a therapeutic patch, its manufacture and use wherein the patch comprises: a fabric layer made of synthetic fibers stretchable in both directions along a substantially orthogonal transverse axis of the patch, an adhesive layer on said face side of the base layer, an active agent dispersed in said adhesive layer, and optionally, a penetration enhancing agent in said adhesive layer.

### BACKGROUND OF THE ART

Transdermal and topical patches and masks represent well-established means for sustained release of therapeutic agents. Satisfactory adhesion of the patch to the skin is directly linked to the efficacy, quality, and safety of the therapeutic treatment. Reduction in the surface area of contact as a result of patch lift, or even the patch falling off, diminishes the delivery of therapeutic ingredient from the patch. Poor adhesion can result in improper dosing of patients. It is well known that current patches detach several times during use.

Generally speaking, adhesion is guaranteed by a specialized class of materials called ‘pressure-sensitive adhesives’ (PSAs) that are defined as adhesives capable of bonding to surfaces with the application of light pressure and, when removed, do not leave any visually noticeable residues. A PSA can be used as main constituent of the formulation (i.e., it serves as a carrier for the active ingredient, assures the control of drug release and, at the same time, confers adhesion properties to the dosage form) or merely added to assure the intimate contact between the dosage form and the skin. Patches can be classified as matrix (drug-in-adhesive) systems, or reservoir, or membrane-controlled systems.

Many aqueous base patches have thick plasters because they contain moisture; therefore, aqueous base patches can be difficult to attach to the skin for long durations. Furthermore, the vaporization of moisture from the patches can cause changes in adhesion and physical properties. Aqueous based preparations are typically significantly heavier in weight and thickness vs non aqueous patches. Aqueous based preparations can have poor adhesive properties. In addition, many ingredients within the adhesive matrix are difficult to dissolve in water and thus not completely dissolved in aqueous patches. Aqueous based patches are heavier and thicker than non-aqueous patches. The thickness and weight can impact movement and may rub on clothing, increasing the likelihood of peeling/detaching from the skin.

The stretch characteristics are extremely important for a topical/transdermal patch applied to moving joints and muscles. Human skin, muscles, and joints can stretch or flex up to 180 degrees and if a patch doesn't stretch greater than or equal to the application area it will restrict movement or detach. Equally important to a fabric's stretch characteristics is its ability to recoil back into shape repeatedly without deformation.

Elastic therapeutic tapes on the market generally utilize 2-way stretch fabric. The two-way (longitudinal only) stretching tapes and patches are either 100% cotton or include up to 5% spandex. These tapes can generally stretch to about 140% of their relaxed length and do not stretch transversely (latitudinal direction) which often creates limitation when performing complex movements and results in higher detachment rates. 4-way stretch tapes are stretchable in both transverse and longitudinal directions. Current 4-way stretch therapeutic tapes on the market generally stretch up to 180% of their relaxed length in the longitudinal direction and up to 75% in the transverse direction and are composed of polyester and spandex. Stretch limitations in the transverse direction can restrict full range of movement, comfortability, and are more prone to detachment. These tapes generally have higher recoil force. Recoil or rebound force is the resistance generated when a fabric is stretched. When the recoil force of a stretched fabric is too high, the patch will fail and detach, or it will restrict movement and not be comfortable to wear.

Furthermore, these 2-way and 4-way stretch therapeutic tapes and patches have low coat weights and are prone to content uniformity issues due to manufacturing limitations. Low coat weights are problematic when including higher quantities of therapeutic agents required to deliver efficacy. Coating weight non-uniformities are major concerns for any patch that delivers therapeutic agents, especially drug ingredients.

The current therapeutic tape design therefore exhibits disadvantage for delivery efficacious quantities of therapeutic agents. Clearly it would be advantageous to provide an improved highly elastic 4-way stretch patch for delivery of therapeutic agents that alleviates at least some of the abovementioned disadvantages of current stretchable therapeutic tape technology, or to at least provide the public with a useful choice.

### SUMMARY OF THE INVENTION

The invention relates to therapeutic patches comprising:
1. a fabric layer stretchable in both directions along a longitudinal axis of the patch and stretchable in both directions along a transverse axis of the patch,
2. an adhesive layer on said face side of the base layer,
3. an active agent dispersed in said adhesive layer, and optionally
4. a skin penetration enhancing agent in said adhesive layer
wherein said adhesive layer attaches the patch to the skin of the user and provides sustained release of the active agent to the skin. The fabric layer comprises nylon, and an elastic material such as spandex (e.g., 5-30%). The patch is stretchable to at least 150% or at least 200% of a relaxed length of the patch in both the longitudinal and transverse axis of the patch. The fabric layer is a woven fabric, advantageously warp-based knit. The patch further comprises a release liner configured to cover the exposed surface of the adhesive layer. The fabric layer comprises between 70% and 95% nylon, e.g., 80% nylon.

In an advantageous embodiment, the adhesive layer is an acrylic based adhesive, and optionally an acrylic based additive. Advantageously, the adhesive layer is an acrylic based adhesive containing copolymers of butyl and 2 ethyl hexyl acrylates. In other embodiments, the adhesive layer is a silicone-based or hydrocolloid-based adhesive.

Active agents of the adhesive layer can include topical pain-relieving agents such as lidocaine, menthol, hemp oil extract or CBD, and capsaicin. Other active agents of the invention include topical antibiotics, prescription, and over-the-counter drugs.

Skin care agents such as hemp oil extract or CBD, hyaluronic acid, ceramides, and collagen can be used in the patches of the invention. Other skin care agents which can be incorporated are one or more of: anti-wrinkle or skin-tightening agents; anti-aging agents; moisturizing agents; skin brightening or depigmentation agents; anti-inflammatory agents; anti-acne agents; DNA repair agents; skin lipid barrier repair agents; anti-cellulite agents; wound-healing agents; stretch-mark/scar removing agents; plumping agents; hair growth retardation agents and hair growth stimulating agents: dark circle reduction or de-puffing agents; collagen synthesis or blood circulation enhancing agents; antioxidants; sebum-controlling agents; pore-minimizing agents, and skin detox or exfoliation agents.

Skin penetration enhancing agents are optionally included in the patches of the invention in the adhesive layer. These agents can include essential oils and terpenes such as d-limonene, menthol/peppermint oil and eucalyptus. Other penetration enhancing agents useful include piperine such as tetrahydropiperine (THP), surfactants such as polysorbate 80, fatty acids such as oleic acid. Lastly, a skin metabolism inhibitor such as Fluvastatin, or a physical enhancer that causes stripping or hydration of the stratum corneum can be added to the adhesive.

The invention is a patch configured to be applied to the skin of a user, the patch comprising:
1. a fabric layer having a face side and a back side, the fabric layer being woven, the yarns or threads being composed of a combination of nylon and spandex,
2. an adhesive layer on the face side of the fabric layer for attaching the patch to the skin of the user,
3. an active agent dispersed in said adhesive layer, and optionally
4. a skin penetration enhancing agent dispersed in said adhesive layer.

The patch can include a film or paper, fluorosilicone, silicone, or non-silicone coated release liner. The invention also includes a method of manufacturing a therapeutic patch comprising the steps of:
1. forming a fabric layer by weaving (e.g., warp and weft knit) nylon material, with an elastic material (e.g., spandex),
2. laminating a layer of adhesive containing an active agent and optionally a penetration enhancing agent on the face side of the formed fabric layer. Optionally, the method includes the step of adhering a release liner on the layer of adhesive for covering the layer of adhesive.

The invention also includes a method of manufacturing a therapeutic patch comprising the steps of:
1. transfer coating the adhesive formula onto a release liner using a knife over roller system,
2. coating an adhesive formula at a 4 mil thickness or greater with a uniform coating, over the entire substrate, within specification,
3. using fans to air dry or moving the coated release liner through an oven until dry, where all solvents and water are evaporated and cross linking or fusing occurs to form an adhesive layer,
4. laminating a 4 way stretch fabric onto the coated release liner using custom web guides that control fabric tension and movement along the transverse direction during the lamination process
5, or optionally, through a multi-layer lamination process, laminating an adhesive barrier layer to the 4 way stretch fabric prior to laminating the coated release liner to form a coated fabric,
6. curing the coated fabric for 48-120 hours,
7. kiss cut or die cutting coated fabric into the patch shapes,
8. adding a perforation(s) or kiss cut(s) to the liner of the coated fabric,
9, and placing coated fabric into a heat seal bag,
10. or optionally placing coated fabric on a corrugated core and winding to create a roll.

The invention further includes a method of treating pain in a subject in need of pain relief comprising applying the therapeutic patch to the subject wherein said active agent is a pain-relieving agent. Additionally, included is a method of delivering a pain-relieving agent to a subject, said method comprising:
applying a patch comprising:
   1. fabric layer made of synthetic fibers stretchable in both directions along a substantially orthogonal transverse axis of the patch,
   2. an adhesive layer on said face side of the fabric layer, and
   3. a pain-relieving agent and optionally a penetration enhancing agent dispersed in said adhesive layer, to a skin surface of said subject; and maintaining said patch on said skin surface for a period of time sufficient for said pain relieving agent to be delivered to said subject.

The invention further includes a method of treating the skin in a subject in need of skin care treatment comprising applying the therapeutic patch to the subject wherein said active agent is a skin care agent. Additionally, included is a method of delivering a skin care agent to a subject, said method comprising:
applying a patch comprising:
   1. fabric layer made of synthetic fibers stretchable in both directions along a substantially orthogonal transverse axis of the patch,
   2. an adhesive layer on said face side of the fabric layer, and
   3. a skin care agent and optionally a penetration enhancing agent dispersed in said adhesive layer, to a skin surface of said subject; and maintaining said patch on said skin surface for a period of time sufficient for said skin care treatment agent to be delivered to said subject.

### DETAILED DESCRIPTION OF THE INVENTION

The subject invention relates to highly elastic patches for delivery of an active agent to the skin. According to the United States Pharmacopeia, ‘transdermal systems’ are designed to deliver the drug(s) through the skin to the systemic circulation. As used herein, the term “patches” includes ‘masks,’ ‘plasters,’ and ‘tapes,’ that deliver a therapeutic agent topically, or transdermally—i.e., allowing systemic administration of the active agent(s).

The patches of the invention are typically thin, lightweight and like a second skin. They can be applied to highly contoured parts of the body including joints and are customizable. They can be cut to any size to accommodate different pain points on the body. Four critical properties of a patch that will determine how effective it is include:
1. The stretch capabilities of the patch substrate/fabric and its ability to expand and contract in both the transverse and longitudinal directions along with the skin.
2. The adhesive strength when applied to the skin and its ability to withstand detachment.
3. The thickness and uniformity of the adhesive to provide higher levels of active agents for efficacy.
4. The adhesive ability to release active and/or inactive ingredients, and its compatibility to the skin.

The subject patches are highly elastic, breathable, water resistant, and skin friendly. They are designed to be a second layer of skin and expand and contract along with the skin without restricting freedom of movement. In advantageous embodiments, the patches comprise a fabric made of nylon and spandex (elastane or lycra) and have an acrylic adhesive matrix (drug/ingredient in adhesive) coating. The patches are self-adhesive due to the adhesive, e.g., acrylic layer. The patches are designed to be similar in thickness and elasticity as the dermis of the skin. The patches are stretchable in all directions (4-way stretch), i.e., in both directions along a longitudinal axis and stretchable in both directions along a transverse axis.

The elasticity of the patches can reach 200%, which is greater than or comparable with the elasticity of human muscles and joints. The patches, with the adhesive coating layer, is placed on a protective paper or film backing in order to protect the adhesive coating. The patches can be dyed any color and cut into any shape/size. The patches contour to any body part or joint without friction on clothing and risk of detaching. The patches permit sustained release of any active and/or inactive agents to the skin, and have strong compatibility with the skin. In advantageous embodiments, the patches permit the active and/or inactive agents to penetrate into the skin effectively through the use of one or more penetration enhancing agents.

The subject therapeutic patches comprises: a fabric layer made of synthetic fibers, stretchable in both directions along a longitudinal axis and stretchable in both directions along a transverse axis, an adhesive layer deposited on said face side of the fabric layer, an active agent dispersed in said adhesive layer, and optionally a skin penetration enhancing agent dispersed in said adhesive layer.

### The Fabric

The fabric of the patch of the invention is substantially deformable and stretchable with great elastic recovery, but sufficiently elastic along longitudinal and transverse axes. The therapeutic patch is typically an elongate strip of material (cut from a roll of tape for instance). The patch is elastic in both directions along a longitudinal axis and elastic in both directions along a transverse axis. In other words, the patch is stretchable in four orthogonal directions along the major plane of a major face of the patch. To give it its stretchable and elastic properties, the fabric layer is composed of an elastic material such as an elastomer and a stretchable fabric, such as nylon. In an advantageous embodiment, the fabric layer is composed of a nylon, as well as spandex or similar material.

Nylon is a robust, stretchy fabric that's incredibly durable. It's super-stretchy and can rebound to its original shape when stretched. Nylon has great elastic recovery; it returns to its original length immediately after stretching. Unlike every other fabric that loses its shape whenever stretched, nylon can return to its shape and stay firm throughout its lifespan. Also. Nylon fabrics are woven to have a degree of stretchability that makes them softer, lighter, and more comfortable to wear. This quality allows most nylon fabrics to be stretched easily both in a lengthwise and widthwise direction without restricting movement. Nylon fabrics are waterproof. Nylon is softer, stronger and stretches more vs polyester or cotton. Both cotton and polyester fibers do not stretch. Nylon has the highest elastic limit of all polymers.

The stretch characteristics are extremely important for a topical/transdermal patch applied to moving joints and muscles. Human skin, muscles, and joints can stretch or flex over 150 degrees and if a patch doesn't stretch greater than or equal to the application area it will restrict movement or detach.

Equally important to a fabric's stretch characteristics is the impact of recoil or rebound force of an elongated patch applied to moving joints and muscles. Recoil or rebound force is a fabrics ability to stretch great percentages in all directions with minimal recoil or rebound force. When the recoil force of a stretched fabric is too high, the patch will fail and detach, or it will restrict movement and not be comfortable to wear. RPET and polyester stretch fabrics have higher recoil force compared to nylon stretch fabrics.

To achieve the desired level of elasticity the fabric layer is advantageously composed of a combination of nylon, and spandex. In an advantageous embodiment, the fabric layer comprises between approximately 70% and approximately 95% by weight of nylon, and between 5% and approximately 30% by weight of spandex, more advantageously between approximately 75% and approximately 82% by weight of nylon, and between approximately 18% and approximately 25% by weight of spandex, and most advantageously approximately 80% by weight of nylon, and approximately 20% by weight of spandex.

In an advantageous embodiment, the fabric layer of the tape is woven from yarns of nylon and spandex. The yarns are woven or knit via weft and warp, or any other type of weave known in the art of fabric production for stretchable fabrics. The warp threads utilize both spandex and nylon. The weft threads utilize both spandex and nylon. The weave is warp-based, meaning the warp, or lengthways threads, are dominant. They are formed into loops and interwoven with weft threads in different ratios to create a lengthways rib pattern on the fabric front and a crosswise rib on the back. This is what gives the material its individual texture and durability.

If the density of the fabric is too low it becomes too floppy and difficult to handle and apply. In advantageous embodiments, the fabric comprises a density of between approximately 170 gsm and approximately 300 gsm, even more advantageously between approximately 190 gsm and approximately 250 gsm, and most advantageously approximately 200 gsm.

Also, in an advantageous embodiment, the patch comprises a fabric layer formed from a yarn of nylon of a grade of between approximately 50D and approximately 120D, more advantageously between approximately 65D and approximately 105D. Whilst the yarn of spandex in the fabric layer is of a grade of between approximately 20D and approximately 60D, more advantageously between approximately 30D and approximately 50D and most advantageously approximately 40D.

The fabrics utilized in the patches of the invention advantageously are made of:
75-90% nylon, and
10-25% spandex.

In an advantageous embodiment, the fabric is 80% nylon and 20% spandex, 90% nylon and 10% spandex, 85% nylon and 15% spandex, or 75% nylon and 25% spandex, 190 gsm-210 gsm (advantageously 200 gsm), and is woven material. Advantageously, the step of weaving comprises weft weaving and warp weaving the nylon material with spandex. Most advantageously the base layer is woven with threads being warp-based, or warp-dominant. The stretch capability of the fabric is 150-200+%, e.g., greater than 150%, greater than 175%, or greater than 200%.

### Advantageous Fabric Specifics:

Nylon 6 or advantageously Nylon 6.6.
Nylon Denier Rating of 40D to 120D, more advantageously between 60D and 100D.

### Spandex, Lycra or Elastane

Denier Rating of 15D to 70D, more advantageously between 20D and 60D.

### The Adhesive

The patch of the invention has a strong tack property when applied to the skin. Tack relates to the ability of an adhesive to form the initial bond with the skin on brief contact under light pressure.

The patch also has strong shear adhesion, or holding power, with the skin. Shear adhesion or shear resistance is defined as the ability to resist flow/movement when shear forces are applied. For a patch to perform well, the shear adhesion or shear resistance property has to guarantee that the adhesive will remain attached to the skin for a specific period of time despite stresses caused by both body movements and cloth frictions.

Further, the patch of the invention can have a low to high peel strength, depending on the area of application and use. Peel Strength relates to its ability to resist removal by peeling. Finally, the peeling-off procedure should be easy and painless, without leaving patch residues and causing skin damage. The patch is safe and gentle to remove from the skin.

The adhesive can be an acrylic based adhesive, or any other form well known in the art of patch technology and it can be applied/coated to the fabric layer via any suitable method. In an advantageous embodiment, a medical grade acrylic-based, silicone-based, hydrocolloid-based, or hydrogel-based adhesive is used. The adhesive can be a heat sensitive adhesive or advantageously, a pressure sensitive adhesive. The adhesive can be applied evenly and uniformly over the entire or a substantial portion of the surface of the fabric layer, or alternatively in regions or intervals such as in transverse and uniformly spaced strips.

PSAs are classified according to their chemical structure (see Venkatraman S, Gale R. Skin adhesives and skin adhesion. 1. Transdermal drug delivery systems. Biomaterials 1998; 19 (13): 1119-36) or the physical form in which they are supplied. In the latter case, PSAs can be categorized as solvent based (non-aqueous), water based, and hot melt.

Three major categories of PSAs are acrylic-based PSAs, silicone-based PSAs, and polyisobutylenes (PIBs). Other materials which can be used in the patches of the invention include polyurethane, hydrocolloids, and hydrogels. Hydrocolloid PSA's are often used for acne treatment and wound dressings that are occlusive and adhesive and can form a gel with water. Hydrogel dressings have similar properties in a gel consistency. Various hydrocolloid gels and dressings have been used in wound management to maintain moisture and aid in debridement of necrotic tissue. Hydrogels contain large amounts of water and matrices that acquire adhesive properties as a result of their moisture content. The adhesive selected for use in the subject invention is chosen based on the particular application and active agent being delivered.

### PIB-Based Adhesives

PIB-based adhesives (PIB-PSAs) can be compounded by blending high- and medium-molecular-weight PIBs, or adding low-molecular-weight polybutylene to this blend. The former formulation is characterized by low peel adhesion values, which decrease as the percentage of the medium-molecular-weight PIB increases. In the latter, the use of low-molecular-weight polybutylene permits to expand the formulation range of the PIB blends conferring to the matrix adhesive properties in terms of tack and peel adhesion.

The main disadvantages in using PIBs are related to their easy oxidation and low air and water vapor permeability. The latter feature can be favorably exploited to enhance the drug flux through the skin; on the other hand, the skin maceration can occur, especially when the patch remains in the same position for prolonged period of time.

### Silicon-Based Adhesives

Silicon-based PSAs are made up of a long chain polymer (polydimethyl siloxane) and a silicate resin. The resin has a high glass transition, while the polymer has a notably low glass transition. The raw material is provided as a mixture of these components and the adhesive properties of the final product depend on their ratio. Since the silanols of such PSAs are susceptible to react with amines, several products have been trimethylsilylated to improve the chemical compatibility and, therefore, patch stability in the presence of cationic drugs and excipients. The silicon-based PSAs excel in drug diffusivity.

### Acrylic-Based Adhesives

Acrylic-based PSAs are obtained by combining ‘hard’ and ‘soft’ monomers at different ratios in order to tune up the final characteristics of the polymer. A third monomer can also be added to improve cohesive properties of the matrix. The large variety of substituted monomers (Table below) allows the incorporation of specific functional groups into the acrylic-based adhesives as well as the synthesis of polymers having versatility in physicochemical properties. Due to the presence of saturated functional groups, the acrylic-based PSAs are more resistant to oxidation with respect to PIB-PSAs; moreover, they are colorless, transparent and do not turn yellow on exposure to sunlight.

Monomers in common use for the preparation of
acrylic pressure-sensitive adhesive copolymers and
their glass transition (T_{g}) values.

| Soft monomers | T_{g} (° C.) | Hard monomers | T_{g} (° C.) |
|---|---|---|---|
| Butyl acrylate | −54 | Methyl methacrylate | 105 |
| Isobutyl acrylate | −40 | Vinyl acetate | 29 |
| 2-Ethyl hexyl acrylate | −85 | Styrene | 100 |
| Ethyl acrylate | −22 | Acrylonitrile | 100 |

### Acrylic-Based Dry Adhesive

In an advantageous embodiment of the invention, the adhesive used in the patch of the invention is a dry solvent based (non-aqueous) adhesive and includes an adhesive base as well as adhesive additive.

### Adhesive Base

The adhesive base is a medical grade solvent based acrylic adhesive contain:
Acrylate monomers
Tackifiers. Tackifiers are usually resins included in the adhesive base
Solvents. The solvents within our adhesive based include ethyl acetate, methyl ethyl ketone, and isopropanol. All solvents are completely removed during the manufacturing process when they are placed through the heat tunnel to cure the product. The coated product is tested for residual solvent levels before it is approved for final conversion and packaging. Solvent based adhesives are more compatible with most ingredients and provide a more uniform coating. Solvent based adhesives also dry faster allowing for quicker curing times and higher outputs.
In an advantageous embodiment, the acrylic adhesive base contains copolymers of butyl and 2 ethyl hexyl acrylate.

### Adhesive Additive

In an advantageous embodiment, an adhesive additive is added to the adhesive base formula to make the peeling-off procedure easier and painless, without leaving residues and causing skin damage.

Medical grade solvent based acrylic adhesive contain:
Acrylate monomers.
Tackifiers. Tackifiers are usually resins included in the adhesive base
Solvents. The solvents within our adhesive additive include ethyl acetate, isopropyl alcohol, naphtha, and methanol. All solvents are completely removed during the manufacturing process when they are placed through the heat tunnel to cure the product. The coated product is tested for residual solvent levels before it is approved for final conversion and packaging.
Solvent based adhesives are more compatible with most ingredients and provide a more uniform coating. Solvent based adhesives also dry faster allowing for quicker curing times and higher outputs.

Both the acrylic adhesive base (high peel strength) and the acrylic adhesive additive (low peel strength) contain copolymers of butyl and 2 ethyl hexyl acrylate. They are both the same composition chemically. The acrylic adhesive additive (low peel version) is held in the reactor longer to create higher molecular weight polymer and has more melamine crosslinker than the higher peel version. They can be blended in any ratio to achieve peel strengths between 2 and 32 oz/in., advantageously 20 or better.

The relative amounts of adhesive base to adhesive additive can be: 25-75% adhesive base and 25-75% adhesive additive. In an advantageous embodiment, the relative amounts are: 75% adhesive base and 25% adhesive additive.

### Active and Inactive Ingredients

The patches of the subject invention can include one or more of the active agents:

### Prescription and OTC Drug Active Ingredients

Fentanyl
Buprenorphine
Daytrana (transdermal Ritalin)
Nicotine
Antianginal (e.g., nitroglycerin)
Anti-Depressants or anti-psychotics (e.g., Selegiline, Mirtazapine, Ensam)
Amphetamines (e.g., Dextroamphetamine. Lisdexamfetamine)
Anti-Nausea (e.g., Promethazine, Scopolamine)
Estrogen and Testosterone
Contraceptive Medication (e.g., Estrogen, Progestin)
Blood Pressure Medication (e.g., Clonidine)
Alzheimer's Treatment (e.g., Donepezil, Rivastigmine)
Anorectal Preparations (e.g., Lidocaine, Hydrocortisone)
Antiseptic and Germicides (e.g., Chlorhexidine, Povidone Iodine)
Dermatological Agents (e.g., Lidocaine, Calamine)
Topical Acne Agents (e.g., Clindamycin, Benzoyl Peroxide, Salicylic Acid, Sulfur)
Topical Analgesics (e.g., Menthol, Capsaicin)
Topical Anesthetics (e.g., Lidocaine, Fentanyl)
Topical Anti-Infectives (e.g., Docosanol, Imiquimod)
Topical Anti-Rosacea Agents (e.g., Azelaic Acid, Metronidazole, Brimonidine)
Topical Antibiotics (e.g., Sodium Fusidate, Mupirocin)
Topical Antifungals (e.g., Clotrimazole, Fluconazole)
Topical Antihistamines (e.g., Doxepin, Diphenhydramine)
Topical Antineoplastic (e.g., Fluorouracil, Imiquimod)
Topical Antipsoriatics (e.g., Betamethasone, Calcipotriene, Tazarotene)
Topical Antivirals (e.g., Penciclovir, Acyclovir)
Topical Astringents (e.g., Witch Hazel)
Topical Debriding Agents (e.g., Collagenase, Balsam Peru, Castor Oil, Trypsin)
Topical Depigmenting Agents (e.g., Fluocinolone, Hydroquinone, Tretinoin)
Topical Emollients (e.g., Emollients, Urea)
Topical Keratolytic (e.g., Podofilox, Salicylic Acid)
Topical Non-Steroidal Anti-Inflammatories (e.g., Diclofenac)
Topical Photochemotherapeutic (e.g., 5-Fluorouracil, Diclofenac)
Topical Rubefacient (e.g., Salicylates, Nicotinate Esters, Capsaicin)
Topical Steroids (e.g., Clobetasol, Diflorasone, Amcinonide, Betamethasone, Desoximetasone, Fluocinonide, Halcinonide)
Topical Steroids with Anti-Infectives (e.g., Aloe Vera, Hydrocortisone, Iodoquinol, Nystatin, Triamcinolone, Acyclovir)

The patches of the subject invention can include one or more of the following:

### Other Active Ingredients, Inactive Ingredients & Cosmetic Ingredients

Acrylate Copolymer
Activated Charcoal
Agave Extract
AHA Fruit Acids
Albizia Flower Extract
Algae Extract
Allantoin
Almond Oil
Aloe Barbadensis (aloe vera)
Alpha Arbutin
Alpha Olefin Sulfonate
Alpha-Hydroxy Acid
Aluminum Chlorohydrate
Aluminum hydroxide
Amaranthus Seed Extract
Amla Oil
Amodimethicone
Anthemis nobilis Flower Extract
Apigenin
Apple Fruit Water
Apricot Kernel Oil
Argan Oil
Argania spinosa Kernel Oil
Argireline (ACETYL HEXAPEPTIDE-8)
Arnica montana flower extract
Arrowroot Starch
Artichoke Leaf Extract
Arugula Leaf Extract
Ashwagandha Extract
Avena sativa (Oat) Kernel Flour (Colloidal Oatmeal)
Avobenzone
Avocado
Bacillus Ferment
Bakuchi Fruit Extract
Bakuchiol
Bamboo Extract
Baobab Oil
Behentrimonium
Behenyl Behenate
Bentonite
Benzocaine
Benzophenone-4
Benzoyl Peroxide
Benzylalcohol-DHA
Beta Glucan
Beta-Hydroxy Acid (BHA)
BHT
BioJelly
Bismuth Oxychloride
Black cohosh
Black Tea Extract
Blood Orange
Blue Flax Extract
Boron
Boswellia
Brassica Alcohol and Glycerides
Brassica Oil Copolymer
Broad Spectrum CBD
Butylene Glycol
C12-15 Alkyl Benzoate
Caffeine
Calamine
Calcium Carbonate
Calendula
Camelina Oil
Camellia Sinensis Leaf Extract
Camphor
Candelilla Wax
Cannabidiol
Cannflavin (e.g., A and B)
Caprylhydroxamic Acid GG
Caprylic/capric Triglycerides
Caprylyl Glycol
Capsaicin
Carbomer
Carnauba Wax
Carrot Cells
Carrot Oil & Beta-Carotene
Castile Soap
Castor Oil
CBD
Ceramides
Ceteareth-20
Ceteareth-25
Cetearyl Alcohol
Cetrimonium Chloride
Cetyl Alcohol
Cetyl Palmitate
Chamomilla recutita (Matricaria) Flower Extract
Chamomile
Chaparral Extract
Charcoal
Chaste Tree Berry (Vitex)
Chickpea Extract
Chlorphenesin
Citric Acid
Citronella Oil
Citrus Combo
Clay
Clove Oil
Cocamidopropyl Betaine
Cocamidopropyl Hydroxysultaine
Cocamidopropylamine Oxide
Coco Betaine
Coco Caprylate Caprate
Coco Glucose
Cocoa butter
Coconut Oil
Coconut Water
Coenzyme Q10 (CoQ10)
Coffee Seed Extract
Collagen
Collagen Protein, Hydrolyzed
Colloidal Oatmeal
Comfrey Root Extract
Cranberry Fruit Water
Cranberry Seed Oil
Cucumber Fruit Extract
Curcumin
Cyclomethicone
Cyclopentasiloxane
d-limonene
Dead Sea Mud
Decyl Glucoside Sodium Lauroyl Lactylate
Diclofenac
Dihydroxyacetone (DHA)
Diisooctyl Succinate
Dimethicone
Dipropylene glycol,
Disodium Edta
DMDM Hydantoin
Edelweiss
EDTA
EGCG (green tea)
Elastin
Emu Oil
Erythrulose
Ethoxydiglycol
Ethylhexyl Palmitate
Evening Primrose
Ferulic Acid
Flavonoids (e.g., flavones, flavonols)
Ginger Root Extract
Ginkgo biloba Leaf Extract
Gluconolactone SB
Glucose-D
Glycan Booster Peptide
Glycerin
Glyceryl Oleate
Glyceryl Stearate
Glycine-Benzoic Acid
Glycol Distearate
Glycol Stearate IP
Glycolic Acid
Glycoproteins
Goji Berry Extract
Goldenseal Extract
Gotu Kola Extract
Grapefruit Water
Grape Seed Extract
Grapeseed Oil
Green Tea Extract
HE-Cellulose, Modified
Hectorite
Hemp Oil Extract and Multiple Constituents Thereof
Hemp Seed Oil
Hemp Seed Protein
Henna Extract
Hexanediol CG
Homosalate
Honey Extract
Honeysuckle Blend
Horse Chestnut Extract
HP Starch
Humectants including aloe, glycerin, hyaluronic acid, propylene glycol, and silicone
Hyaluronic Acid (high mol. weight)
Hyaluronic Acid (low mol. weight)
Hyaluronic Acid (medium mol. weight)
Hydrocolloid
HydroComplex
Hydrogenated Polyisobutene
Hydrolyzed Hyaluronic Acid
Hydroxypropyl Guar
Hydroxypropyl Methylcellulose
Hyssop Extract
Irish Moss Extract
Isododecane
Isoeicosane
Isohexadecane
IsoLanolin
Isopropyl Myristate
Isopropyl Palmitate
Jojoba
Kakadu Plum Extract
Kaolin Clay
Kelp Extract
Keratin Protein, Hydrolyzed
Knotgrass Flavonoids
Kojic Acid
Lactic Acid
Lacto-Ceramide
Lanolin
Laureth-3
Lauryl Laurate
Lavender
Lavender Essential Oil
Lecithin
Lemon balm extract (Melissa officinalis)
Lentil Extract
Licorice Root
Lidocaine
Lingonberry Stem Cells
Lupine Protein, Hydrolyzed
Lychee Extract
Lycii Berry Extract
Macadamia Nut Oil
Magnesium Aluminum Silicate
Magnesium chloride
Magnesium Stearate
Mallow Extract
Mango Butter, USDA Certified Organic
Manuka Honey
Marrubium Extract
Marshmallow Root Extract
Marula Tetradecane
Meadowfoam Seed Oil
Menthol
Methyl Gluceth-10
Methyl Salicylate
Methylsulfonylmethane (MSM)
Milk Protein, Hydrolyzed
Mineral Oil
Mulberry Root Extract
Murumuru Butter
Myristic Acid
Myristyl Myristate
Natural Bisabolol
Natural Ferulic Acid
Natural Peptide
Neroli Hydrosol
Nettle Extract
Niacinamide
Oat
Oatmeal Extract
Octocrylene
Octyldodecanol
Orange Peel Butter
Stem Cells
Oxybenzone
Ozokerite Wax
Palmitic Acid
Palmitoyl
Panthenol
Papaya Enzymes
Paraben-DU
Paullinia Cupana Seed Extract
Pea Extract
Pearl Powder
PEG-150 Distearate
PEG-40 Hydrogenated Castor Oil
PEG-7 Glyceryl Cocoate
PEG-8 Beeswax
PEG-8 Dimethicone
Pentylene Glycol
Peppermint Oil
Persia gratissima (Avocado) Oil
Petroleum Jelly (Petrolatum)
Phenoxyethanol
Phenylpropanol EHG
Phyto Ceramides
Pineapple Enzymes
Plankton Extract
Polyamide 3
Polybutene
Polyethylene
Polyglucose
Polyglyceryl Oleate
Polyhydroxystearic Acid
Polyisobutene
Polymethylsilsesquioxane
Polyphenols
Polyquaternium
Polysilicone
Polysorbate
Potassium Sorbate
Pramoxine
Propanediol
Propylene Glycol
Protein-Hyaluronate Blend
Provitamin B5 (d-panthenol)
Pumice Powder
Pumpkin Puree
PVP (Polyvinylpyrrolidone)
Quaternium-31
Quercetin
Quinoa Protein, Hydrolyzed
Radish Root Ferment Filtrate
Red Raspberry Seed Oil
Repair VITA Oil
Resveratrol
Retinol
Rhodiola
Rhubarb Root Extract
Rice Bran Beads
Rice Quat
Rice Starch
Rose Essence Water
Rose Flower Extract
Rose Hip Oil
Rosemary
Rosemary Leaf Extract
Saccharomyces
Sage Extract
Salicylic Acid
Sea Buckthorn
Sea Fennel
Sea Kelp
Sea Whip
Sesame Seed Oil
Shea Butter
Shea Oil
Silica
Silicone
Simmondsia chinensis (Jojoba) Seed Oil
Sodium Benzoate
Sodium Hyaluronate
Sodium Hydroxide
Sorbitan Stearate
Sorbitol
Soy-Rice Peptides
Squalane
Squalene
Stearic Acid
Stearoxy Trimethylsilane
Stearyl Alcohol
Stearyl Palmitate
Sucrose Cocoate
Sucrose Stearate
Sugarcane Extract
Sulfosuccinate
Sulphur Mud
Sunflower Oil
Sunflower Wax
Tapioca Starch
Tara Gum Gel
Tea Tree Oil
Teprenone
Titanium Dioxide
Tocopheryl Acetate
Tomato Lycopene
Tribehenin
Triethanolamine
Triglyceride
Trihydroxystearin
Tripeptide-5
Urea
Vitamin A
Vitamin B12
Vitamin B3
Vitamin C
Vitamin E
Walnut Shell Powder
Water
Watermelon Extract
Wheat Protein, Hydrolyzed
White Curcumin
White Tea Extract
Willow Bark Extract
Witch Hazel
Wrinkle Blur
Yerba Mate Extract
Yogurt Filtrate
Zinc

### Penetration Enhancers for Topical and Transdermal Delivery

The patches of the invention optionally includes penetration enhancers, (also referred to as permeation enhancers), dispersed in the adhesive layer. Penetration enhancing agents permit the active and/or inactive agents to penetrate into the skin effectively by transiently enhancing skin permeability without damaging viable cells.

The permeation enhancers selected should possess the following properties: pharmacologically inert, non-irritating, non-toxic, non-allergenic, compatible with the active and/or inactive agents, have good solvent properties, odorless, tasteless, colorless, and allow the skin to quickly regain to its natural barrier.

The two major categories of permeation enhancers for transdermal and topical ingredient delivery are chemical/synthetic permeation enhancers and natural permeation enhancers. In an advantageous embodiment of the invention, natural essential oils, and terpenes such as d-limonene, menthol/peppermint oil and eucalyptus are used. Other natural permeation enhancers of the invention include fatty acids such as oleic acid and piperine such as tetrahydropiperine (THP).

### Chemical Permeation Enhancers

Chemical permeation enhancers are molecules that interact with the constituents of skin's outermost and rate limiting layer, the stratum corneum, and increase its permeability. Chemical based permeation enhancers are synthetic and include alcohols (ethanol, 2-propanol, caprylic alcohol), sulphoxides (dimethyl sulphoxide, dimethylacetamide), azone (1-dodecylazacycloheptan-2-one, laurocapran), pyrrolidones (2-pyrrolidone, N-methyl-2-pyrrolidone), urea, fatty acids and derivatives (lauric acid, myristic acid, caprylic acid, oleic acid), polyols (propylene glycol, glycerol), surfactants (ionic: SLS and non-ionic: polysorbates), chelating agents (EDTA, citric acid) polyols (propylene glycol, glycerol), surfactants (ionic: SLS and non-ionic: polysorbates), and chelating agents (EDTA, citric acid).

### Natural Permeation Enhancers

Natural permeation enhancers work by changing the structure of the stratum corneum barrier and interaction with intercellular stratum corneum lipids to increase diffusivity of active and/or inactive agents. Polarity, molecular weight (<500 Da), concentration of active and/or inactive compounds in formulation, solubility of molecules in oil and water and composition of preparation significantly affect their penetration through the skin. Therefore, only a minority of molecules with specific physio-chemical properties can cross the skin sufficiently. Among them are essential oils and their active constituents (terpenes, terpenoids). Essential oils and terpenes (primarily extracted from essential oils) have been widely investigated as safe and suitable skin permeation enhancers for both hydrophilic and hydrophobic ingredients. Other natural permeation enhancers include piperine such as tetrahydropiperine (THP) and cosmoperine.

The permeation enhancers of the invention for transdermal and topical ingredient delivery can vary based on the area of application and sensitivity of skin in that specific area of the body. The anatomical structure of skin (thickness, composition of intercellular stratum corneum lipids, number of skin shafts, density of hair follicles, vascular anatomy, and collagen fiber arrangement) differs between people and different areas of the body. Those differences in the structure of the skin affect the quantity and ease of penetration of the active and/or inactive agents through the skin. For example, the skin on the face is thinner and more susceptible to irritation compared to other areas of the body with thicker skin, like the back. For a face application, a natural permeation enhancer with low irritation is used, such as d-limonene.

In an advantageous embodiment, a skin permeation enhancer is added to the adhesive base formula to allow the active and/or inactive agents to penetrate into the skin effectively. Advantageously, the permeation enhancer is a natural permeation enhancer containing essential oils or terpenes. More advantageously, the natural permeation enhancer contains d-limonene, menthol/peppermint oil, or eucalyptus. In other embodiments, the permeation enhancer includes fatty acids such as oleic acid, piperine such as tetrahydropiperine or surfactants such as polysorbate 80.

### Natural Permeation Enhancers Include:

Essential Oils:
Ajuput
Alpinia oxyphylla
Anise
Basil
Black Cumin
Cardamom
Chamomile
Chenopodium
Citronella
Clove
Eryngium Bungei
Eucalyptus
Fennel
Ginger
Lavender
Lilacin
Melissa
Mentha
Menthol
Myrtle Oils
Niaouli
Nutmeg
Orange
Peppermint
Petit Grain
Rosemary
Sage
Tea Tree
Thyme
Tulsi
Turpentine
Ylang

### Terpenes:

Anethole
α-Bisabolol
Borneol
Camphor
Carvacrol
Carvone
1,8-Cineole
1,4-Cineole
Cymene
Eugenol
Farnesol
Fenchone
Geraniol
Limonene
Linalool
Menthol
Menthone
Nerolidol
α-Pinene oxide
Pulegone
Rose oxide
Safranal
Terpinen-4-ol (4-terpinenol)
α-Terpineol
Tetra-hydrogeraniol
Thymol
Valen-cene
Verbenon-e

### Fatty Acids:

Oleic
Linoleic
Palmitoleic
Palmitic
Stearic

### Piperine:

Tetrahydropiperine
Cosmoperine

### Method of Producing the Patch

There are several challenges and limitations when manufacturing a highly elastic patch with therapeutic agents. To have a high enough concentration of active ingredients in the adhesive matrix, the adhesive thickness, (i.e., coat weight) must be high and uniform throughout. Coating weight non-uniformities are one of the major loss categories in the web-coating process. Because the performance of the coating is a function of the actual coating weight, a uniform coating at the desired coating weight, over the entire substrate, is essential to producing a saleable quality product. The coating weight must be uniform within a roll, down-web and cross-web and give an average coating weight that is within specification. If the coating weight, uniformity, and roll average vary outside of limits, the product will not function as intended and will be scrapped, resulting in a yield loss, and increasing costs. Furthermore, adding a higher concentration of active ingredients without increasing the coat weight is extremely problematic. By doing so the percent of adhesive is reduced in the adhesive matrix, greatly affecting the adhesive characteristics. Tack, peel force and shear adhesion are greatly reduced to the point the product would not be suitable to be worn for long periods of time, on joints or during movement activities without peeling off. Reports to FDA reveal that Ineffective adhesion is a significant problem.² When patches lose adherence, patients may get less medication.

As someone trained in the art would understand, to overcome some of these coating challenges, a transfer coating technique is utilized to manufacture a web of multiple layers utilizing different adhesive formulas. These specific compounded formulas are applied to a release paper or film and then laminated to fabric; The coated web is then carried through an oven to dry, fuse or cross link the formulas to provide an impervious smooth surface. The major advantage of this process is that coating film (i.e., release liner) is suspectable to less defects before it is applied to the fabric. This technique is used for fabrics that are stretchy and can't be coated by direct coating method. Because they would distort under the tension applied to obtain a flat surface and the adhesive formula matrix would sink into knitted fabric and fabric stiffing would occur resulting in less fabric stretch and loss in tear strength.

Transfer coating has the unique benefit of being able to coat higher weights of adhesive compared to all other adhesive technologies. However, coating anything over 3 mil thickness often results in content uniformity issues across the web. Stretchable sports tapes and Kinesiology tapes are not subject to these challenges due to their low coat weights which are often 1-3 mil in thickness. For most applications, 4 ml and higher coat weights are required to provide therapeutic quantities of active ingredients. Coat weights of 4 mil or greater and are subject to these content uniformity challenges.

To ensure proper content uniformity, a knife over roll technique is used. The knife over roll system has a much higher accuracy vs other coating methods, and combined with the transfer coating method, can coat higher weights of adhesive. In the knife over roll system configuration, a suitably designed doctor blade is properly positioned on top of a high-precision roller. The gap between the bottom of the blade and the thickness of the fabric that passes over the roller controls primarily the coating weight. The roll may be a rubber covered or more advantageously, chromium-plated steel roll. Steel rolls can give more precise coat weights. The viscosity of the formula matrix directly affects the coat weight. Higher viscosity is required to produce a higher coat weight. Materials with a wide range viscosity (up to 40,000 cps) can be coated by the knife on roll technique.

The last critical step in the coating process is laminating the fabric to the coating film. A person skilled in the art will understand that good tension control of the web entering the lamination nip is of extreme importance for maintaining consistent and high quality in coating and laminating processes. For example, in coating, variation in web tension will lead directly to variations in coat weight and poor final product bonding. If either web is under tensioned, issues like wrinkle lines, blemishes, and fold-overs may occur in the web and show in the finished product. Too much tension may result in issues such as stretch lines in the web which may show up in the product. Additionally, too much tension in one of the webs may result in web curl in the finished product.

Laminating a highly stretchable fabric creates challenges for tension control over the web. Proper and consistent tension is critical to ensure no blemishes occur, and most importantly to ensure accurate content uniformity. Web guides on all current coating systems were not designed to control lateral (i.e., transverse) tension of a highly stretchable fabric that moves in both latitudinal and longitudinal directions. 4-way stretch fabric moves in latitudinal and longitudinal directions. Fabric that stretches in transverse direction will shift regardless of the web guides being utilized and regardless of the tension control thus creating content uniformity and in some cases blemishes. To overcome this, specially designed web guides were created to control fabric tension and movement along the transverse direction during the lamination process.

The finished product contains higher coat weights (4 mil or above), precise content uniformity, efficacious quantities of therapeutic delivery agents, proper bonding of the fabric layer to the adhesive matrix, and adhesive that is void of blemishes.

Patches of the invention can be made by preparing and mixing the adhesive formula as outlined in the batching procedure (see Examples below). The adhesive formula is then applied to the release liner. The coated liner is carried through an oven until dry, where all solvents and water are evaporated, and cross linking or fusing of copolymers of adhesive formula occurs. The 4 way stretch fabric is then laminated to the coated liner. The coated fabric is left to cure for 48-120 hours. When applicable, liner is then printed. Fabric is kiss cut or die cut into the patch shapes. A Perforation(s) or kiss cut(s) is then added to the liner. Product is placed into heat seal bags and sealed before final packaging occurs.

### Methods of Using the Patches of the Invention

The patches of the invention are highly versatile and can incorporate a wide variety of agents (see active agent list above).

An advantageous embodiment of the invention are pain relief patches.

### Pain Relief Patches

Pain relief patches of the invention comprise:
1. a fabric layer stretchable in in both directions along a longitudinal axis and stretchable in both directions along a transverse axis,
2. an adhesive layer on said face side of the base layer,
3. a pain-relieving agent dispersed in said adhesive layer, and optionally
4. a penetration enhancing agent dispersed in said adhesive layer.

Advantageously the fabric used for the pain relief patch is 80%-90% nylon and 10%-20% spandex, 190 gsm-210 gsm, the weave is warp-based, the warp and weft threads are composed of both spandex and nylon.

### Adhesives

Advantageous adhesives used in the pain relief patches are:
Polyacrylate Copolymers (Acrylics)
Polysiloxanes (Silicones)
Polyisobutylenes (PIBs)
Hot Melt
Urethanes
Hydrocolloids
Hydrogels

### Active Agents

Active agents of a pain relief patch can include one or more of:
Allantoin
Aluminum Acetate
Benzocaine
Calamine
Camphor
Capsaicin or Capsicum
Cupric Sulfate
Dexpanthenol
Dibucaine
Dibucaine Hydrochloride
Diclofenac Sodium
Eucalyptus Oil
Glycol Salicylate
Hemp oil extract or individual constituents thereof, e.g., Cannabinoids (125 compounds including CBD, CBN, CBC, CBG), Phenols (42 compounds including Spiro-Indans, Dihydrostilbenes, Dihydrophenanthrenes, Simple Phenols), Flavonoids (34 compounds including apigenin, quercetin, luteolin, vitexin, isovitexin, orientin), or Terpenes (120 compounds including myrcene, alpha-pinene, beta-pinene, caryophyllene, geraniol, humulene, limonene, linalool, eucalyptol)
Histamine Dihydrochloride
Hydrocortisone
Lidocaine
Menthol
Methyl Salicylate
Nonivamide
Peppermint Oil
Petrolatum
Phenol
Pramoxine Hydrochloride
Sulfur
Trolamine Salicylate
Wintergreen Oil
Zinc Acetate
Zinc Oxide

### Inactive Agents

Inactive agents of the patch optionally include one or more of:
Alcohol
Aloe Barbadensis (Aloe Vera) Leaf Juice
Arnica Montana Flower Extract
B-12 (Methylcobalamin)
Black Cohosh
Boswellia
Calendula
Chamomile
Chaste Tree Berry (Vitex)
D-Limonene
Dipropylene Glycol
Eucalyptus
Folate/Folic Acid
GABA
Hemp oil extract or individual constituents thereof, e.g., Cannabinoids (125 compounds including CBD, CBN, CBC, CBG), Phenols (42 compounds including Spiro-Indans, Dihydrostilbenes, Dihydrophenanthrenes, Simple Phenols), Flavonoids (34 compounds including apigenin, quercetin, luteolin, vitexin, isovitexin, orientin), or Terpenes (120 compounds including myrcene, alpha-pinene, beta-pinene, caryophyllene, geraniol, humulene, limonene, linalool, eucalyptol)
L-Theanine
Lavender
Lemon Balm
Magnesium Chloride
Melatonin
Methylsulfonylmethane (MSM)
Passionflower
Polybutene
Polysorbate 80
Potassium Sorbate
Sodium Benzoate
Tocopheryl Acetate (Vitamin E)
Vitamin B-6
Vitamin C
Water, And
White Curcumin

### Penetration Enhancing Agents

Penetration enhancing agents of a pain relief patch can include One or more of:
Essential Oils (Chamomile, Eucalyptus, Melissa, Menthol, Orange, Peppermint, Rosemary, Tea Tree)
Terpenes (Camphor, Limonene, Menthol, Menthone, Rose oxide, Thymol)
Piperine (Tetrahydropiperine, Cosmoperine)

### Examples of Pain Relief Patches are as Follows:

Pain Relief Patch #1

| | | | | Permeation | | | |
|---|---|---|---|---|---|---|---|
| Active Agent | Qty | Adhesive | Qty | Enhancer | Fabric | Qty | Liner |
| Lidocaine | 4% | Acrylic | 89% | Menthol | Nylon/Spandex | 80%/20% | Paper |

Pain Relief Patch #2

| | | | | Permeation | | | |
|---|---|---|---|---|---|---|---|
| Active Agent | Qty | Adhesive | Qty | Enhancer | Fabric | Qty | Liner |
| Menthol | 6% | Acrylic | 92% | Limonene | Nylon/Spandex | 80%/20% | Film |

Pain Relief Patch #3

| | | | | Permeation | | | |
|---|---|---|---|---|---|---|---|
| Active Agent | Qty | Adhesive | Qty | Enhancer | Fabric | Qty | Liner |
| Lidocaine | 4% | Acrylic | 85% | Menthol | Nylon/Spandex | 80%/20% | Paper |

Pain Relief Patch #4

| | | | | Permeation | | | |
|---|---|---|---|---|---|---|---|
| Active Agent | Qty | Adhesive | Qty | Enhancer | Fabric | Qty | Liner |
| Menthol | 6% | Acrylic | 82% | Limonene | Nylon/Spandex | 80%/20% | Paper |

### Skin Care Patches

Skin care patches of the invention comprise:
1. a fabric layer stretchable in in both directions along a longitudinal axis and stretchable in both directions along a transverse axis,
1. an adhesive layer on said face side of the base layer,
2. an active agent dispersed in said adhesive layer and optionally,
3. a penetration enhancing agent dispersed in said adhesive layer.

Advantageously the fabric used for the pain relief patch is 80%-90% nylon and 10%-20% spandex, 190 gsm-210 gsm, the weave is warp-based, the warp and weft threads are composed of both spandex and nylon.

### Adhesives

Advantageous adhesives that are used in the skin care patches of the invention include:
Polyacrylate Copolymers (Acrylics)
Polysiloxanes (Silicones)
Polyisobutylenes (PIBs)
Hot Melt
Urethanes
Hydrocolloids
Hydrogels

### Active Agents

Active agents of the skin care patch can include one or more of:
Activated Charcoal
Allantoin
Aloe Vera
Alpha Arbutin
Apigenin
Argireline (ACETYL HEXAPEPTIDE-8)
Bakuchiol
Bentonite & Kaolin Clay
Chamomile Oil
Clove Oil
Collagen (hydrolyzed)
CoQ10 (ubiquinol)
EGCG (green tea)
Evening Primrose
Hemp oil extract or individual constituents thereof, e.g., Cannabinoids (125 compounds including CBD, CBN, CBC, CBG), Phenols (42 compounds including Spiro-Indans, Dihydrostilbenes, Dihydrophenanthrenes, Simple Phenols), Flavonoids (34 compounds including apigenin, quercetin, luteolin, vitexin, isovitexin, orientin), or Terpenes (120 compounds including myrcene, alpha-pinene, beta-pinene, caryophyllene, geraniol, humulene, limonene, linalool, eucalyptol)
Hyaluronic Acid (high mol. weight)
Hyaluronic Acid (low mol. weight)
Hydrocolloid
Lemon balm extract (Melissa officinalis)
Licorice Root
Manuka Honey
Natural Salicylic Acid
Niacinamide
Papaya & Pineapple Enzymes
Phyto Ceramides
Plant Based Squalane
Rosehip Oil
Rosemary.
Sea Buckthorn Oil
Sea Whip
Silicone
Tea Tree Oil
Vitamin A
Vitamin B12
Vitamin B3
Vitamin C
Vitamin E
White Curcumin
Witch Hazel
Zinc

The skin care agents used in the skin care patches of the invention are selected from the group consisting of:
Anti-Wrinkle or Skin-Tightening Agents, (e.g., Argireline, Bakuchiol, Sea Buckthorn Oil)
Anti-Aging Agents (e.g., Coq10, Phyto Ceramides, Collagen, Hyaluronic Acid)
Moisturizing Agents (e.g., Allantoin, Phyto Ceramides, Squalane, Rosehip Oil, Sea Whip, Evening Primrose)
Skin Brightening or Depigmentation Agents (e.g., Niacinamide, Alpha Arbutin)
Anti-Inflammatory Agents (e.g., Aloe Vera, Hemp Extract Oil or CBD, Chamomile Oil, EGCG, Lemon Balm, Licorice Root)
Anti-Acne Agents (e.g., Clove Oil, Tea Tree Oil, Witch Hazel, White Curcumin)
Dna Repair Agents (e.g., Photolysomes, Mitosomes, Endosomes)
Skin Lipid Barrier Repair Agents (e.g., Vitamin E, Phyto Ceramides, Apigenin)
Anti-Cellulite Agents (e.g., Coleus Forskohlii, Caffeine, Boswellia, Horse Chestnut, Amarantus, Olives, Black Pepper)
Wound-Healing Agents (e.g., Manuka Honey, Silicone, Aloe Vera, Vitamin B12)
Stretch-Mark/Scar Removing Agents (e.g., Manuka Honey, Silicone, Vitamin A)
Plumping Agents; (e.g., Cupuacu Butter, Honey, Hyaluronic Acid, Vitamin C)
Hair Growth Retardation Agents and Hair Growth Stimulating Agents (e.g., Aloe Vera, Ginseng, Onion, Rosemary, Honey, Hyaluronic Acid, Vitamin C, Melatonin)
Dark Circle Reduction or De-Puffing Agents (e.g., Kojic Acid, Vitamin E, Peptides, Arnica, Retinol)
Collagen Synthesis or Blood Circulation Enhancing Agents (e.g., DMAE, MSM, Retinoids, Alpha Hydroxy Acids, Beta Hydroxy Acids, Epidermal Growth Factor)
Antioxidants (e.g., Zinc, Niacinamide, Glycolic Acid)
Scbum-Controlling Agents (e.g., Vitamin A, Hydrocolloid)
Pore-Minimizing Agents (e.g., Lactic Acid, Red Clover, Ribose)
Skin Detox or Exfoliation Agents (Salicylic Acid. Bentonite & Kaolin Clay, Activated Charcoal, Willow Bark, Papaya & Pineapple Enzymes)

### Penetration Enhancing Agents

Penetration enhancing agents of skin care patches can include one or more of:
Essential Oils (Chamomile, Clove, Eucalyptus, Melissa, Menthol, Orange, Peppermint, Rosemary, Tea Tree)
Terpenes (Camphor, Limonene, Menthol, Menthone, Rose oxide, Thymol)
Fatty Acids (Oleic, Linoleic, Palmitoleic, Palmitic, Stearic)
Piperine (Tetrahydropiperine, Cosmoperine)

Examples of skin care patches are as follows:

Skin Care Patch #1

| | | | Permeation | Liner |
|---|---|---|---|---|
| Active Ingredients | Adhesive | Fabric | Enhancer | Type |
| Apigenin, Argireline, | Silicone | Nylon/ | Limonene | Fluoro- |
| Bakuchiol, Collagen, | | Spandex | | silicone |
| CoQ10 (ubiquinol), | | | | |
| EGCG (green tea), | | | | |
| Evening Primrose, | | | | |
| Hyaluronic Acid (high | | | | |
| and low molecular | | | | |
| weight), Niacinamide, | | | | |
| Phyto Ceramides, | | | | |
| Plant Based Squalane, | | | | |
| Sea Buckthorn Oil, | | | | |
| Vitamin E | | | | |

Skin Care Patch #2

| | | | Permeation | Liner |
|---|---|---|---|---|
| Active Ingredients | Adhesive | Fabric | Enhancer | Type |
| Allantoin, Aloe Vera, | Silicone | Nylon/ | Limonene | Fluoro- |
| Alpha Arbutin, Apigenin, | | Spandex | | silicone |
| Bakuchiol, Chamomile | | | | |
| Oil, Collagen, CoQ10 | | | | |
| (ubiquinol), EGCG | | | | |
| (green tea), Evening | | | | |
| Primrose, Hyaluronic | | | | |
| Acid (low molecular | | | | |
| weight), Lemon | | | | |

Skin Care Patch #3

| | | | Permeation | Liner |
|---|---|---|---|---|
| Active Ingredients | Adhesive | Fabric | Enhancer | Type |
| Activated Charcoal, | Hydrocolloid | Nylon/ | Limonene | Film |
| Alpha Arbutin, | | Spandex | | |
| Bakuchiol, Bentonite | | | | |
| Clay, Kaolin Clay, | | | | |
| Chamomile Oil, Clove | | | | |
| Oil, CoQ10 (ubiquinol), | | | | |
| EGCG (green tea), | | | | |
| Evening Primrose, | | | | |
| Hyaluronic Acid (low | | | | |
| molecular weight), | | | | |
| Lemon Balm Extract, | | | | |
| Licorice Root, Manuka | | | | |
| Honey, Natural Salicylic | | | | |
| Acid, Niacinamide, | | | | |
| Rosehip Oil, Sea Whip, | | | | |
| Tea Tree Oil, Vitamin A, | | | | |
| Vitamin E, Witch Hazel, | | | | |
| Zinc | | | | |

Skin Care Patch #4

| | | | Permeation | Liner |
|---|---|---|---|---|
| Active Ingredients | Adhesive | Fabric | Enhancer | Type |
| Activated Charcoal, | Hydrocolloid | Nylon/ | Limonene | Film |
| Alpha Arbutin, | | Spandex | | |
| Bakuchiol, Bentonite | | | | |
| Clay, Kaolin Clay, | | | | |
| Chamomile Oil, Clove | | | | |
| Oil, CoQ10 (ubiquinol), | | | | |
| EGCG (green tea), | | | | |
| Evening Primrose, | | | | |
| Hyaluronic Acid (low | | | | |
| molecular weight), | | | | |
| Lemon Balm Extract, | | | | |
| Licorice Root, Manuka | | | | |
| Honey, Natural Salicylic | | | | |
| Acid, Niacinamide, | | | | |
| Rosehip Oil, Sea Whip, | | | | |
| Tea Tree Oil, Vitamin A, | | | | |
| Vitamin E, Witch Hazel, | | | | |
| Zinc | | | | |

Skin Care Patch #5

| | | | Permeation | Liner |
|---|---|---|---|---|
| Active Ingredients | Adhesive | Fabric | Enhancer | Type |
| Manuka Honey, Aloe | Silicone | Nylon/ | Limonene | Fluoro- |
| Vera, Vitamin A, | | Spandex | | silicone |
| Vitamin B12, Vitamin E, | | | | |
| Vitamin C, Rosehip Oil, | | | | |
| White Curcumin, | | | | |
| Lavender Oil | | | | |

Skin Care Patch #6

| | | | Permeation | Liner |
|---|---|---|---|---|
| Active Ingredients | Adhesive | Fabric | Enhancer | Type |
| Apigenin, Argireline, | Silicone | Nylon/ | Oleic Acid | Fluoro- |
| Bakuchiol, Collagen, | | Spandex | | silicone |
| CoQ10 (ubiquinol), | | | | |
| EGCG (green tea), | | | | |
| Evening Primrose, Hemp | | | | |
| Oil Extract, Hyaluronic | | | | |
| Acid (high and low | | | | |
| molecular weight), | | | | |
| Phyto Ceramides, | | | | |
| Vitamin E | | | | |

Skin Care Patch #7

| | | | Permeation | Liner |
|---|---|---|---|---|
| Active Ingredients | Adhesive | Fabric | Enhancer | Type |
| Alpha Arbutin, | Hydrocolloid | Nylon/ | Oleic Acid | Film |
| Bakuchiol, Bentonite | | Spandex | | |
| Clay, Kaolin Clay, | | | | |
| Chamomile Oil, Clove | | | | |
| Oil, CoQ10 (ubiquinol), | | | | |
| EGCG (green tea), | | | | |
| Evening Primrose, Hemp | | | | |
| Oil Extract, Hyaluronic | | | | |
| Acid (low molecular | | | | |
| weight), Lemon Balm | | | | |
| Extract, Licorice Root, | | | | |
| Manuka Honey, | | | | |
| Niacinamide, Rosehip | | | | |
| Oil, Tea Tree Oil, | | | | |
| Vitamin A, Vitamin E, | | | | |
| Witch Hazel | | | | |

### Hormone Therapy Patches

Hormone therapy patches of the invention comprise:
1. a fabric layer stretchable in in both directions along a longitudinal axis and stretchable in both directions along a transverse axis,
2. an adhesive layer on said face side of the base layer,
3. a hormone therapy agent dispersed in said adhesive layer, and optionally
4. a penetration enhancing agent dispersed in said adhesive layer.

Advantageously the fabric used for the pain relief patch is 80%-90% nylon and 10%-20% spandex, 190 gsm-210 gsm, the weave is warp-based, the warp and weft threads are composed of both spandex and nylon.

Active Agents are Selected from:

### Active Agents

Estrogen
Conjugated Estrogen
Estradiol
Progestin
Estropipate
Norethindrone Acetate
Levonorgestrel
Anastrozole
Tamoxifen
Letrozole
Progesterone
Testosterone

### Adhesives

Advantageous adhesives that are used in the hormone therapy patches of the invention include:
Polyacrylate Copolymers (Acrylics)
Polysiloxanes (Silicones)
Polyisobutylenes (PIBs)
Hot Melt
Urethanes
Hydrocolloids
Hydrogels

### Penetration Enhancing Agents

Penetration enhancing agents of a hormone therapy patch can include one or more of:
Essential Oils (Chamomile, Clove, Eucalyptus, Melissa, Menthol, Orange, Peppermint, Rosemary, Tea Tree)
Terpenes (Camphor, Limonene, Menthol, Menthone, Rose oxide, Thymol)
Fatty Acids (Oleic, Linoleic, Palmitoleic, Palmitic, Stearic)
Piperine (Tetrahydropiperine, Cosmoperine)

An example of a hormone therapy patch is below.

Hormone Therapy Patch

| | | | | Permeation | | | |
|---|---|---|---|---|---|---|---|
| Active Agent | Qty | Adhesive | Qty | Enhancer | Fabric | Qty | Liner |
| Estradiol | 0.025 mg | Acrylic | 89% | Menthol | Nylon/Spandex | 80%/20% | Film |

### Anti-Depressant Patches

Anti-depressant patches of the invention comprise:
1. a fabric layer stretchable in in both directions along a longitudinal axis and stretchable in both directions along a transverse axis,
2. an adhesive layer on said face side of the base layer,
3. an anti-depressant agent dispersed in said adhesive layer, and optionally
4. a penetration enhancing agent dispersed in said adhesive layer.

Advantageously the fabric used for the pain relief patch is 80%-90% nylon and 10%-20% spandex, 190 gsm-210 gsm, the weave is warp-based, the warp and weft threads are composed of both spandex and nylon.

Advantageous active agents are as follows.

### Active Agents

Selegiline
Mirtazapine

### Adhesives

Advantageous adhesives that are used in the anti-depressant patches of the invention include:
Polyacrylate Copolymers (Acrylics)
Polysiloxanes (Silicones)
Polyisobutylenes (PIBs)
Hot Melt
Urethanes
Hydrocolloids
Hydrogels

### Penetration Enhancing Agents

Penetration enhancing agents of an anti-depressant patch can include one or more of:
Essential Oils (Chamomile, Clove, Eucalyptus, Melissa, Menthol, Orange, Peppermint, Rosemary, Tea Tree)
Terpenes (Camphor, Limonene, Menthol, Menthone, Rose oxide, Thymol)
Fatty Acids (Oleic, Linoleic, Palmitoleic, Palmitic, Stearic)
Piperine (Tetrahydropiperine, Cosmoperine)

An example of an anti-depressant patch is below.

Anti-Depressant Patch

| | | | | Permeation | | | |
|---|---|---|---|---|---|---|---|
| Active Agent | Qty | Adhesive | Qty | Enhancer | Fabric | Qty | Liner |
| Selegiline | 6 mg | Acrylic | 89% | Menthol | Nylon/Spandex | 80%/20% | Film |

The following Examples are illustrative, but not limiting of the compounds, compositions, and methods of the present invention. Other suitable modifications and adaptations of a variety of conditions and parameters normally encountered which are obvious to those skilled in the art are within the spirit and scope of this invention.

### EXAMPLES

### Example 1

Mixing Preparation for Pain Patch with Lidocaine

The batching takes place in a suitable kettle equipped with propeller mixer agitation. A batch can be made at room temperature; no heating is required. In a suitable side vessel with propeller agitation, charge ingredients in the following order, allowing each to dissolve/disperse before adding the next:
Alcohol (where indicated)
Water (where indicated)
Arnica Extract
Boswellia Extract
MSM
Aloe Leaf Juice
Calendula Extract
White Curcumin
Polysorbate 80
Tocopheryl Acetate
Lavender Oil
Menthol (where indicated)
Sodium Benzoate (where indicated)
Potassium Sorbate (where indicated)
Hemp Oil Extract (where indicated)

Add adhesive (e.g., acrylic) to mixing drum. Verify the weight of the adhesive in the drum and start mixer at 70%.

Carefully add in active agent (e.g., Lidocaine) and increase mixer to 100%. Allow to mix for 30 minutes after active agent is added.

Add polybutene (where indicated) and mix slowly.

Add the alcohol dispersion of ingredients to the liquid adhesive base in the main vessel and mix to incorporate.

Allow to mix for at least 30 minutes, or until completely dissolved. 17

### Patch Production

1. Mixing preparation and completion of the adhesive formula
2. Liner and Fabric are prepared for coating and lamination
3. Full width Liner and fabric are 58″ wide or greater

Transfer coating of the adhesive formula matrix on the release liner. The coater spreads the adhesive matrix formula using knife over steel roll or knife over rubber rolls. The coater can accommodate a wide variety of coat weights and thickness, from 0.5 mils to 100 mils.

In-Line coating thickness measurement to ensure coating thickness accuracy.

The coated liner is carried through an oven to dry, fuse or cross link the formulas to provide an impervious smooth surface.
4. Lamination of the 4 way stretch fabric onto the coated liner while carefully controlling the web tension in the transverse direction.
5. Splicing and Slitting as needed.
6. Finished coated fabric is slit to 14″ wide rolls.
7. Wind up of the finished coated fabric.
8. Finished coated fabric is wrapped, sealed, and transported for conversion.
9. During the conversion process the coated fabric is placed on rewind/unwind machine for quality checks.
10. Qualified coated fabric is placed in the Mark Andy Die Cutting and Printing Press for printing on the release liner and Die Cutting.
   a) Optionally, the liner is printed using a printing pattern.
11. Rotary Dies are used to perforate or kiss-cut the release liner. A Micro-perforation or kiss-cut is added down the center of the liner.
   a) Optionally, horizontal, and vertical perforations are added between each strip.
12. Rotary Dies are used to kiss cut or die cut into the patch shapes.
   a) Optionally, the product is slit into the appropriate length (10 strips or 5 patches).
13. The product is placed into a heat seal bag and sealed using a heat seal machine.
   a) Optionally, the product is put on a tall, corrugated core with 1″ inside diameter.
   b) Optionally, an adhesive tab is placed on the core and liner. The purpose of the tab is to keep the product in place while it is wound onto the core.
   c) Optionally, the product is wound to the core and a final adhesive tab is placed on the end of the liner. The purpose of the tab is to ensure the roll doesn't unwind prior to final packaging. This tab is resealable and can be resealed by the end consumer as needed after use.
   d) Optionally, shrink wrap is added to the finished roll. Shrink wrap has double vertical perforations for easy removal. Shrink wrap is placed in a heat or steam tunnel to shrink to size around finished roll.
14. Heat sealed bag containing the finished product is placed into a folding carton and closed.
   a) Optionally, a finished roll is inserted into a HDPE bottle.
   b) Optionally, a CR (child resistant) cap is applied and tightened onto bottle.
   c) Optionally, a shrink sleeve is added to bottle.

### Example of Lidocaine Patch:

| Ingredient | % of wet adhesive | % of dry adhesive |
|---|---|---|
| Adhesive base | 94.05 | 84.63 |
| Lidocaine HCl, monohydrate | 1.45 | 4.20 |
| Hemp Oil Extract | 2.33 | 6.77 |
| Arnica Montana Extract | 0.01 | 0.04 |
| White Curcumin | 0.01 | 0.04 |
| Boswellia Extract | 0.01 | 0.04 |
| MSM | 0.01 | 0.04 |
| Aloe Barbadensis Leaf Juice | 0.01 | 0.04 |
| Tocopheryl Acetate (Vitamin E) | 0.00 | 0.01 |
| Calendula Extract Oil | 0.01 | 0.04 |
| Lavender Extract Oil | 0.18 | 0.53 |
| Polysorbate 80 | 0.03 | 0.09 |
| L-Menthol | 1.88 | 3.55 |

### Example 2

Pain Patch with Menthol

Batching procedure takes place in a suitable kettle equipped with propeller mixer agitation. Batches can be made at room temperature; no heating is required.

In a suitable side vessel with propeller agitation, charge ingredients in the following order, allowing each to dissolve/disperse before adding the next:
Alcohol
Black Cohosh extract (where indicated)
Chaste Tree Berry Extract (where indicated)
White Curcumin
Magnesium Chloride
Lavender Oil (where indicated)
Lemon Balm (where indicated)
Eucalyptus Oil (where indicated)
Chamomile Oil (where indicated)
Tocopheryl Acetate
d-limonene
Dipropylene glycol
Polysorbate 80
Hemp Oil Extract (where indicated)
Vitamin B-12 (where indicated)
Vitamin B-6 (where indicated)
Folate/Folic Acid (where indicated)
Vitamin C (where indicated)
Arnica Extract (where indicated)
Melatonin (where indicated)
Passionflower (where indicated)
GABA (where indicated)
L-Theanine (where indicated)

Add adhesive (e.g., acrylic) to mixing drum. Verify the weight of the adhesive in the drum and start mixer at 70%.

Carefully add in active agent (e.g., menthol) and increase mixer to 100%. Allow to mix for 30 minutes after OTC drug is added.

Add the alcohol dispersion of ingredients to the liquid adhesive base in the main vessel and mix to incorporate.

Allow to mix for at least 30 minutes, or until completely dissolved.

Using a patch production similar to that described in Example 1, an example of a menthol patch product is:

### Ingredients

| Ingredient | % of wet adhesive | % of dry adhesive |
|---|---|---|
| High Peel Adhesive base | 67.53 | 62.62 |
| Low Peel Adhesive base | 22.51 | 20.87 |
| White Curcumin | 0.02 | 0.04 |
| Tocopheryl Acetate (Vitamin E) | 0.02 | 0.04 |
| Polysorbate 80 | 0.04 | 0.09 |
| Magnesium Chloride | 0.02 | 0.04 |
| d-limonene | 0.08 | 0.18 |
| Dipropylene Glycol | 0.08 | 0.00 |
| Arnica Montana Extract | 0.08 | 0.19 |
| I-Menthol | 4.13 | 6.04 |
| Lavender Extract Oil | 0.12 | 0.28 |
| Chamomile Extract Oil | 0.08 | 0.19 |
| Melatonin | 0.20 | 0.46 |
| Passionflower | 0.33 | 0.76 |
| GABA | 0.33 | 0.76 |
| L-Theanine | 0.33 | 0.76 |
| Isopropyl Alcohol | 1.22 | 0.00 |
| Hemp Oil Extract | 2.88 | 6.68 |

It will be readily apparent to those skilled in the art that numerous modifications and additions can be made to both the present compounds and compositions, and the related methods without departing from the invention disclosed.

### Advantages

The present invention provides a number of advantages over what is currently in use for transdermal and topical delivery of therapeutic agents. The invention is a 4-way stretch therapeutic tape that stretches in both the transverse and longitudinal directions and stretches at least 200% allowing for a better consumer experience, especially when used on moving joints and the face.

The present invention is fully customizable and can be used anywhere on the body. It is well suited for use for longer durations of time and is not restricted for use on a specific body part only (i.e., lower back). As a skincare or acne treatment patch or mask, the present invention can stay on the face all day/night while exercising, talking, smiling, eating, and sleeping. As a pain relief patch, the present invention can stay on a moving joint or muscle all day/night and will not detach while performing sports, exercise, and daily activities.

In order to achieve these advantages a therapeutic patch has been devised which is composed of a stretch nylon and spandex fabric, compared to that of known patches that are predominantly cotton, polyester or nonwoven materials. The therapeutic patch of the present invention can stretch to over 200% of its resting length and has great elastic recovery, rebounding to its original shape even after repeated use. The patch has low recoil force preventing detachment without restricting movement. Unlike known patches with higher recoil force that will fail, detach, restrict movement and not be comfortable to wear.

The therapeutic patch of the present invention is comprised of woven elastic and nylon threads in both the warp and weft direction and the weave is warp-based. This is what gives the material its stretch characteristics, individual texture and durability. Unlike known patches, which are jersey knit, weft-based or nonwoven and have zero or limited transverse stretch and prone to detachment.

The therapeutic patch of the present invention has a higher coat weight, with uniform coating at the desired coating weight, over the entire substrate, within specification. Known therapeutic tapes have low coat weights and are prone to content uniformity issues due to manufacturing limitations. Low coat weights are problematic when including higher quantities of therapeutic agents required to deliver efficacy. Coating weight non-uniformities are major concerns for any patch that delivers therapeutic agents, especially drug ingredients.

The foregoing description of the invention includes preferred forms thereof. Modifications may be made thereto without departing from the scope of the invention as defined by the accompanying claims.

What is claimed is:

1. A therapeutic patch comprising:
a) a fabric layer stretchable in both directions along a longitudinal axis of the tape and stretchable in both directions along a transverse axis of the tape,
b) an adhesive layer on said face side of the base layer, and
c) an active agent and optionally a penetration enhancing agent dispersed in said adhesive layer, wherein said adhesive layer attaches the patch to the skin of the user and provides sustained release of the active agent to the skin.

2. A therapeutic patch as in claim 1 wherein the fabric layer comprises nylon, and an elastic material.

3. A therapeutic patch as in claim 1 wherein the elastic material is spandex.

4. A therapeutic patch as in claim 1 wherein the patch is stretchable to at least 150% of a relaxed length of the patch.

5. A therapeutic patch as claimed in claim 1 wherein the patch is stretchable to at least 200% of a relaxed length of the patch.

6. A therapeutic patch as in claim 1 wherein the fabric layer is a woven fabric.

7. A therapeutic patch as claimed in claim 1 wherein the fabric weave is warp-based.

8. A therapeutic patch as claimed in claim 1 wherein the fabric layer compromises an elastic warp thread and elastic weft thread.

9. A therapeutic patch as claimed in claim 1 wherein the patch further comprises a release liner configured to cover the exposed surface of the adhesive layer.

10. A therapeutic patch as claimed in claim 9 wherein the release liner is fluorosilicone, silicone or non-silicone coated film or paper.

11. A therapeutic patch as claimed in claim 1 wherein the fabric layer has a density of between 170 gsm and 300 gsm.

12. A therapeutic patch as claimed in claim 11 wherein the fabric layer has a density of 200 gsm.

13. A therapeutic patch as claimed in claim 1 wherein the fabric layer comprises between 70% and 95% nylon.

14. A therapeutic patch as claimed in claim 1 wherein the fabric layer comprises approximately 80% nylon.

15. A therapeutic patch as claimed in claim 1 wherein the elastic material is spandex.

16. A therapeutic patch as claimed in claim 15 wherein the fabric layer comprises between 5% and 30% spandex.

17. A therapeutic patch as claimed in claim 15 wherein the fabric layer comprises approximately 20% spandex.

18. A therapeutic patch as claimed in claim 1 wherein the adhesive coat thickness is 4 mil or greater

19. A therapeutic patch as claimed in claim 1 wherein the adhesive layer has uniform coating at the desired coating weight, over the entire substrate, within specification

20. A therapeutic patch as claimed in claim 1 wherein the adhesive layer is an acrylic based adhesive.

21. A therapeutic patch as claimed in claim 1 wherein the adhesive layer comprises an acrylic base adhesive and an acrylic based additive.

22. A therapeutic patch as claimed in claim 1 wherein the adhesive layer is an acrylic based adhesive containing copolymers of butyl and 2 ethyl hexyl acrylate.

23. A therapeutic patch as claimed in claim 1 wherein the adhesive layer is a silicone-based adhesive.

24. A therapeutic patch as claimed in claim 23 wherein the adhesive layer is a medical grade platinum catalyzed skin safe silicone-based adhesive.

25. A therapeutic patch as claimed in claim 1 wherein the adhesive layer is a hydrocolloid-based adhesive.

26. A therapeutic patch as claimed in claim 25 wherein the adhesive layer is a hydrocolloid-based adhesive containing gel-forming agents.

27. A therapeutic patch as claimed in claim 1 wherein the active agent is lidocaine.

28. A therapeutic patch as claimed in claim 1 wherein the active agent is menthol.

29. A therapeutic patch as claimed in claim 1 wherein the active agent is hemp oil extract or CBD.

30. A therapeutic patch as claimed in claim 1 wherein the active agent is a skin care agent.

31. A therapeutic patch as claimed in claim 1 wherein the active agent is selected from the group consisting of fentanyl, buprenorphine, daytrana, nicotine, antianginal (e.g. nitroglycerin), anti-depressant, anti-psychotic, amphetamine, anti-nausea agent, estrogen, 10 testosterone, contraceptive medication, blood pressure medication, Alzheimer's medication, anorectal preparation, antiseptic, germicide, dermatological agent, acne agent, analgesic, anesthetics, anti-infective, anti-rosacea agent, antibiotic, antifungal, antihistamine, antineoplastic, antipsoriatic, antiviral, astringent, debriding agent, depigmenting agent, emollient, keratolytic, non-steroidal anti-inflammatory, photochemotherapeutic, rubefacient, and steroid.

32. A therapeutic patch as claimed in claim 1 further comprising one or more inactive agents in the adhesive layer selected from the group consisting of alcohol, aloe barbadensis (aloe vera) leaf juice, Arnica montana flower extract, black cohosh, boswellia, calendula, chamomile, chaste tree berry (Vitex), d-limonene, dipropylene glycol, Eucalyptus, folate/folic acid, GABA, hemp oil extract, 1-theanine, lavender, lemon balm, magnesium chloride, melatonin, menthol, methylsulfonylmethane (MSM), passionflower, polysorbate 80, potassium sorbate, sodium benzoate, tocopheryl acetate (vitamin E), vitamin B-12, vitamin B-6, vitamin C, water, and white curcumin.

33. A therapeutic patch as claimed in claim 1 further comprising one or more skin penetration enhancing agents in the adhesive layer selected from the group consisting of Essential Oils (Chamomile, Clove, Eucalyptus, Melissa, Menthol, Orange, Peppermint, Rosemary, Tea Tree), Terpenes (Camphor, Limonene, Menthol, Menthone, Rose oxide, 30 Thymol), Fatty Acids (Oleic, Linoleic, Palmitoleic, Palmitic, Stearic) and Piperine (Tetrahydropiperine, Cosmoperine).

34. A therapeutic lidocaine patch as in claim 1 wherein said adhesive layer comprises:
4 wt % lidocaine,
80 to 96 wt. % copolymers of butyl and 2 ethyl hexyl acrylate,
and 0.1 to 1 wt. % polysorbate 80.

35. A therapeutic menthol patch as in claim 1 wherein said adhesive layer comprises:
6 wt % menthol,
80 to 94 wt. % copolymers of butyl and 2 ethyl hexyl acrylate,
0.1 to 1 wt. % polysorbate 80,
and 0.1 to 1 wt. % dipropylene glycol.

36. A method of manufacturing a therapeutic patch as in claim 1 comprising the steps of:
a) forming a fabric layer by weaving nylon with an elastic material,
b) laminating a layer of adhesive containing an active agent and optionally a penetration enhancing agent on the face side of the formed fabric layer.

37. A method as claimed in claim 36 further comprising the step of adhering a release liner on the layer of adhesive for covering the layer of adhesive.

38. A method as claimed in claim 36 wherein the step of weaving comprises weft weaving and warp weaving the nylon material with spandex.

39. A method as claimed in claim 36 further comprising the step of weaving is warp-based.

40. A method of manufacturing a therapeutic patch comprising the steps of:
a) transfer coating the adhesive formula onto a release liner using a knife over roller system,
b) moving the coated release liner through an oven until dry, where all solvents and water are evaporated and cross linking or fusing occurs to form an adhesive layer,
c) laminating a 4 way stretch fabric onto the coated release liner to form a coated fabric using web guides that control fabric tension and movement along the transverse direction during the lamination process
d) and optionally laminating an adhesive barrier layer to the 4 way stretch fabric prior to laminating the coated release liner to form a coated fabric,
e) curing the coated fabric for 48-120 hours,
f) kiss cut or die cutting coated fabric into the patch shapes,
g) adding a perforation(s) or kiss cut(s) to the liner of the coated fabric,
h) and optionally placing coated fabric on a corrugated core, and winding to create a roll.

41. A method of treating pain in a subject in need of pain relief comprising applying the therapeutic patch of claim 1 to the subject wherein said active agent is a pain-relieving agent.

42. A method of delivering a pain-relieving agent to a subject, said method apply a patch comprising:
a) fabric layer made of synthetic fibers stretchable in both directions along a longitudinal axis and stretchable in both directions along a transverse axis
b) an adhesive layer on said face side of the fabric layer, and
c) a pain-relieving agent and penetration enhancing agent dispersed in said adhesive layer, to a skin surface of said subject; and maintaining said patch on said skin surface for a period of time sufficient for said pain relieving agent to be delivered to said subject.
